(19) United States
(12) Patent Application Publication
(10) Pub. No.: US 2021/0172016 A1
(43) Pub. Date: Jun. 10, 2021

(54) DOSAGE AND VARIETAL RECOMMENDATIONS FOR THE TREATMENT OF MEDICAL CONDITIONS USING CANNABIS

(71) Applicant: MedReleaf Corp., Toronto (CA)

(72) Inventors: Angelo Fefekos, Milton (CA); Shannon Theresa Louise O'Hearn, Toronto (CA); George Shawn Charames, Toronto (CA)

(21) Appl. No.: 16/762,289

(22) PCT Filed: Nov. 7, 2018
(86) PCT No.: PCT/CA2018/051406

§ 371 Date: May 7, 2020

Related U.S. Application Data
(60) Provisional application No. 62/582,479, filed on Nov. 7, 2017.
(60) Provisional application No. 62/582,463, filed on Nov. 7, 2017.

(51) Int. Cl. C12Q 1/6883; A61K 31/05

(52) U.S. Cl. CPC C12Q 1/6883; C12Q 2600/156; C12Q 2600/106; A61K 31/05

(57) ABSTRACT

Described are methods for determining a recommended dosage and/or variety of cannabis for a subject based on genetic testing. The presence or absence of genetic variants in a sample from the subject is determined and used to determine a recommended dosage of cannabis, estimate the sensitivity of the subject to cannabis, or select a subject for the treatment of a medical condition. In some embodiments the genetic variants include polymorphisms in or near CYP2C9, CYP3A4 and/or CYP2C19, optionally that are associated with cannabinoid metabolism. The recommended dosage may be for a specific variety of cannabis for treating a medical condition.

### RELATED APPLICATIONS

This application claims the benefit of priority of US Provisional Patent Application No. 62/582,463 and U.S. Provisional patent Application No. 62/582,479, the entire contents of which are hereby incorporated by reference.

### FIELD OF THE INVENTION

The present invention relates to cannabis and more specifically to methods for determining a recommended dosage and/or variety of cannabis for the treatment of a medical condition based on genetic testing.

### BACKGROUND OF THE INVENTION

Cannabis contains over 421 different chemical compounds of several molecular classes, including flavonoids, terpenes, steroids, and cannabinoids. Of these compounds, cannabinoids are perhaps the most well studied group, in particular tetrahydrocannabinol (THC) and cannabidiol (CBD) (ElSohly et al., 2007). Both THC and CBD bind to endogenous cannabinoid receptors of the mammalian endocannabinoid system, resulting in a variety of downstream effects related to the modulation of mood, memory, appetite, pain, and inflammation (Grotenhermen, 2004). Thus, medical cannabis is used in the management of a variety of conditions and symptoms related to these modulatory effects, including pain, nausea and vomiting, depression, anxiety, and insomnia (Ben Amar, 2006; Huestis, 2007; Whiting et al., 2015).

In addition to cannabinoids, terpenes are another important class of compounds thought to be associated with the physiological effects of cannabis. 120 unique terpenes have been isolated in the cannabis plant (ElSohly et al., 2007). Terpenes produce their own range of pharmacological activities, and it is possible that they are equally, if not more important, than cannabinoids in terms of therapeutic effects. The physiological effects of cannabis are a product of the composition and concentration of active constituents found in each variety, as well as their complex pharmacological interactions and metabolism in the human body. In addition, the pharmacokinetics of cannabinoids also varies depending on the route of administration (Huestis, 2007).

Clinical and anecdotal evidence suggest that the delivery of whole-plant products is more effective than isolated cannabinoids for symptom management. This is believed to be a consequence of synergistic interactions between cannabinoids, terpenes, and other plant constituents, often described as the “Entourage Effect” (Russo and Taming, 2011).

While cannabis speciation has been a topic of debate, the current scientific consensus is that it is a single species known as Cannabis sativa L. with three commonly recognized sub-species, indica, sativa, and ruderalis (Hazekamp et al., 2016). Interbreeding of these subspecies has resulted in the creation of over 700 hybrid varieties that span the morphological and pharmacological characteristics of both sativa and indica plants (Hazekamp et al, 2012) Different cannabis hybrids express differing cannabinoid ratios and terpene profiles. Moreover, identical strains grown in different environments may also produce different cannabinoid and terpene profiles. The varying physiological effects induced by indica and sativa strains leads to different clinical uses of cannabis varieties. For example, indica dominant varieties are traditionally associated with more sedative or relaxing effects, while sativa dominant varieties are thought to produce more stimulating or energizing effects (Hazekamp et al., 2012).

Different people respond to cannabis differently. Some individuals appear more or less susceptible to the effects of cannabis and may require a smaller or larger dose in order to obtain a desired therapeutic benefit or avoid any undesirable side-effects. This uncertainty may also deter some individuals from the recreational or therapeutic use of cannabis who would otherwise benefit.

With hundreds of cannabis varieties available, each with its own molecular profile and therapeutic properties, it can be a challenge for individuals and/or healthcare providers to identify a suitable dose and/or variety of cannabis for the treatment of a particular medical condition. Accordingly, there remains a need for methods for determining a recommended dosage and/or variety of cannabis for the treatment of a medical condition.

### SUMMARY OF THE INVENTION

In one aspect, there is provided a pharmacogenetics-based cannabis compatibility test for determining a recommended dose and/or strain of cannabis for the treatment of a medical condition in a subject.

Genetic markers and/or combinations of genetic markers are described that are useful for determining the sensitivity of a subject to cannabis or to specific cannabinoids such as THC or CBD. The corresponding genotype can then be used to recommend a dosage and/or strain of cannabis for a subject, optionally by generating a cannabis compatibility score.

The methods described herein may also be used to provide a recommended dosage and/or strain of cannabis for the treatment of a particular medical condition in a subject. Clinical data shows that different strains of cannabis vary in their efficacy for the treatment of different medical conditions. This variability, coupled with the varying sensitivity of different subjects to the physiological effects of cannabis, can hamper the determination of what strain and/or what dosage of cannabis should be used or recommended to a subject for the treatment of a medical condition. Furthermore, different strains of cannabis can vary significantly in the relative amount of THC and/or CBD they contain. In one embodiment, the methods described herein allow for the determination of a recommended therapeutic dosage (such as grams of bud per day) of a specific variety of cannabis for the treatment of a specific condition.

The methods described herein for determining a recommended dose and/or variety of cannabis for the treatment of a medical condition may also help improve patient and physician confidence in a treatment plan, avoid undesirable side effects and reduce costs.

In one embodiment, the methods described herein may be used to identify subjects that are slow metabolizers of cannabinoids who may benefit from a lower therapeutic dose of cannabis relative to subjects who are fast metabolizers of cannabinoids. In one embodiment, the methods described herein may be used to identify subjects who are fast metabolizers of cannabinoids who may require a higher therapeutic dose in order to obtain similar therapeutic effects relative to subjects who are slow metabolizers of cannabinoids. The methods may also be used to identify subjects who are fast metabolizers of certain cannabinoids and slow metabolizers of other cannabinoids allowing for dosage and/or varietal recommendations to be tailored accordingly. In one embodiment, the methods described herein may be used to determine a recommended dose of cannabis based on the combination of genetic testing of variants associated with cannabinoid metabolism as well as clinical data identifying particular varieties and/or dosages as preferentially efficacious for the treatment of specific medical conditions. Also provided are methods for the treatment of a medical condition comprising determining the presence or absence of genetic variants in a sample from a subject, and identifying the subject for treatment with cannabis based on the presence or absence of genetic variants in the sample.

Accordingly, in one embodiment there is provided a method of determining a recommended dosage and variety of cannabis for treating a subject with a medical condition. In one embodiment, the method comprises:

determining the presence or absence of one or more genetic variants in a sample from the subject;

determining a recommended variety of cannabis for the subject based on the medical condition of the subject; and/or

determining a recommended dosage of the recommended variety of cannabis for the subject based on the presence or absence of the one or more genetic variants in the sample.

In one embodiment, the method further comprises determining the recommended dosage of the recommended variety of cannabis for the subject based on the presence or absence of the one or more genetic variants in the sample and the medical condition of the subject. For example, in one embodiment a higher or lower dose of a variety of cannabis may be recommended to a subject using cannabis for the treatment of symptoms associated with cancer relative to a subject using cannabis for the treatment of anxiety.

In one embodiment, determining the recommended dosage for the subject comprises:

generating a cannabis compatibility score for the subject based on the presence or absence of the one or more genetic variants; and

comparing the cannabis compatibility score to one or more control scores, wherein each control score is associated with a recommended dosage.

As shown in the Examples, a cannabis compatibility score may be generated based on the presence or absence of genetic variants in the sample from the subject. The cannabis compatibility score may then be compared to a control score in order to determine a recommended dosage of cannabis. In one embodiment, the cannabis compatibility score is a THC compatibility score and is indicative of the sensitivity of the subject to THC and/or is useful for determining a recommended dosage of THC. In another embodiment, the cannabis compatibility score is a CBD compatibility score and is indicative of the sensitivity of the subject to CBD and/or is useful for determining a recommended dosage of CBD. In one embodiment, the cannabis compatibility score is a composite score indicative of sensitivity of the subject to THC and CBD.

In one embodiment, the cannabis compatibility control score is compared to one or more control scores associated with a recommended dosage and/or variety for the treatment of a particular medical condition. For example, in one embodiment a CBD compatibility score is generated for a subject with a medical condition based on the presence or absence of genetic variants in CYP2C19 and CYP3A4 and the CBD cannabis compatibility score is compared to a series of control scores associated with dosages of a specific variety of cannabis containing CBD for the treatment of the medical condition.

The medical condition may be any medical condition for which a therapeutic benefit is obtained from the administration of cannabis for the treatment of the medical condition and/or any symptoms associated with the medical condition. For example, in one embodiment the medical condition is anxiety, appetite disorder, depression, inflammation, pain, nausea and/or vomiting, seizures, or a sleep disorder. In one embodiment, the medical condition is cancer or any symptoms associated with cancer. In one embodiment, there is provided a method for identifying a subject responsive to treatment of pain with CBD, optionally with Avidekel, comprising detecting the presence of one or more SNPs in a sample from the subject listed in Table 15. In one embodiment, the SNP is SNP rs734969. In one embodiment, the presence of a heterozygous A allele at SNP rs734969 identifies a subject responsive to treatment with pain.

In one embodiment, there is provided a method for identifying a subject responsive to treatment of pain with THC, optionally with Sedamen of Luminarium, comprising detecting the presence of one or more SNPs in a sample from the subject listed in Table 16. In one embodiment, the SNP is rs1856908. In one embodiment, the presence of a heterozygous T allele at SNP rs1856908 identifies a subject responsive to treatment with pain with THC.

In one embodiment, the sample may be any biological sample that can be assayed for the genetic variants described herein. In one embodiment, the sample comprises nucleic acids such as DNA or mRNA. In one embodiment, the sample is a cheek swab or a blood sample. In one embodiment, the sample comprises genomic DNA. Optionally, the sample may be treated or purified prior to testing the sample for the genetic variants described herein.

In another aspect, there is also provided a method of treating a subject with cannabis. In one embodiment, the method comprises determining a recommended dosage and/or variety of cannabis for treating a subject with a medical condition as described herein and administering the recommended dosage and/or variety of cannabis to the subject. Optionally, the recommended dosage of cannabis is self-administered such as by inhalation or ingestion.

For example, in one embodiment there is provide a method for treating pain in a subject in need thereof, the method comprising detecting the presence of one or more SNP variants listed in Table 15 in a sample from the subject, selecting a subject for treatment with CBD based on the presence or absence of the one or more SNP variants, and administering CBD to the subject selected for treatment with CBD. In one embodiment, the method comprises administering cannabis comprising CBD, optionally Avidekel. In one embodiment, administering CBD comprises self-administration. In one embodiment, the subject is selected form the treatment of pain based on the presence of an A allele at SNP rs734969. In one embodiment, the subject is selected form the treatment of pain based on the presence of a heterozygous A allele at SNP rs734969.

Also provided is the use of CBD for the treatment of pain in a subject in need therefore, wherein the subject has been selected for treatment based on the presence or absence of one or more SNP variants listed in Table 15. In one embodiment, cannabis comprising CBD is used for the treatment of pain in the subject, optionally Avidekel.

In one embodiment there is provide a method for treating pain in a subject in need thereof, the method comprising detecting the presence of one or more SNP variants listed in Table 16 in a sample from the subject, selecting a subject for treatment with THC based on the presence or absence of the one or more SNP variants, and administering THC to the subject selected for treatment with THC. In one embodiment, the method comprises administering cannabis comprising THC, optionally Sedamen or Luminarium. In one embodiment, administering THC comprises self-administration. In one embodiment, a subject is selected for the treatment of pain with THC based on the presence of a T allele at SNP rs1856908. In one embodiment, the subject is selected form the treatment of pain based on the presence of a heterozygous T allele at SNP rs1856908.

Also provided is the use of THC for the treatment of pain in a subject in need therefore, wherein the subject has been selected for treatment based on the presence or absence of one or more SNP variants listed in Table 16. In one embodiment, cannabis comprising THC is used for the treatment of pain in the subject, optionally Sedamen or Luminarium.

In another aspect, there is provided a kit for performing a method as described herein. In one embodiment, the kit is useful for determining a recommended dosage and/or variety of cannabis for a subject. In one embodiment, the kit comprises nucleic acid primers or probes suitable for amplifying and/or genotyping one or more genes and/or genetic variants as described herein. Also provided is the use of nucleic acid primers or probes for amplifying and/or genotyping one or more genes and/or genetic variants as described herein for determining a recommended dosage of cannabis for the treatment of a subject with a medical condition.

In another embodiment, there is provided a system for determining a recommended dosage and/or variety of cannabis for a subject. In one embodiment, the system comprises a processor configured for generating or obtaining a cannabis compatibility score for a subject based on the presence or absence of one or more genetic variants in a sample from the subject, comparing the cannabis compatibility score to one or more control scores, and determining a recommended dosage and/or variety of cannabis for the subject. In one embodiment, the recommended dosage and/or variety of cannabis for the subject is for the treatment of a medical condition.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

Embodiments of the invention will now be described in relation to the drawings in which:

FIG. 1 shows a flow chart of one embodiment of a method for determining a recommended dosage and variety of cannabis for the treatment of a medical condition in a subject.

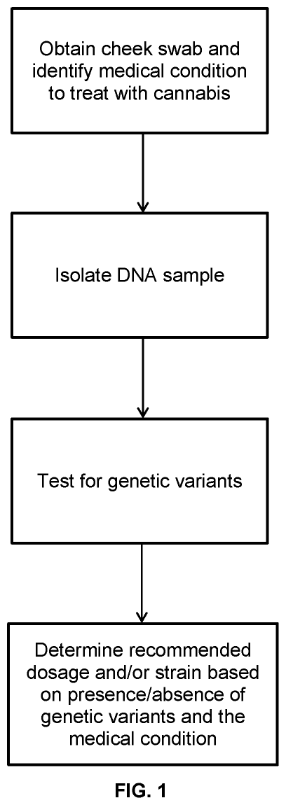

### DETAILED DESCRIPTION OF THE INVENTION

The present description provides methods for determining a recommended dosage and/or variety of cannabis for a subject based on genetic testing. Optionally, the method is for determining a recommended dosage and/or strain of cannabis for treating a subject with a medical condition.

As set out herein, the use of genetic testing allows for the determination of the sensitivity of a subject to the physiological effects of cannabis, and in particular to cannabinoids such as THC and/or CBD. A number of genetic variants have been identified that are associated with the activity and/or expression of enzymes that metabolize THC and/or CBD, including variants in CYP2C19, CYP2C9 and CYP3A4. Determining the presence or absence of these genetic variants may therefore be useful for identifying subjects that metabolize THC and/or CBD relatively quickly and may benefit from or require a higher dose of THC and/or CBD to realize a desired therapeutic effect, or for identifying subjects that metabolize THC and/or CBD relatively slowly and may benefit from or require a lower dose of THC and/or CBD, in order to realize a desired therapeutic effect.

Furthermore, different varieties of cannabis may differ both in the relative amounts of cannabinoids such as THC and/or CBD or other physiologically active compounds, as well as efficacy for the treatment of different medical conditions including symptoms associated with medical conditions. As shown in the Examples, specific varieties of cannabis that are particularly efficacious for the treatment of specific medical conditions have been identified based on survey data of patients who have been prescribed medical cannabis. Combining this clinical data with genetic data allows for the determination of personalized recommended dosage and/or varieties of cannabis for the treatment of a specific medical condition.

As set out in Examples 3 and 4, genetic variants have been identified in subjects with pain who are responsive to treatment with either cannabis comprising CBD (Avidekel) or cannabis comprising THC (Sedamen or Luminarium.

As used herein, “cannabis” refers to plant material from the genus of flowering plants including the species Cannabis sativa, Cannabis indica and Cannabis ruderalis as well as crosses, hybrids and/or variants thereof, that produce detectable levels of cannabinoids as well as extracts and preparations made using cannabis plant material.

As used herein “variety” or “strain” refers to the collective descendants produced from a common ancestor that share a uniform morphological or physiological character. Cannabis varieties or strains may either be pure or hybrid varieties of the Cannabis genus of plants, that encompasses the species C. sativa, C. indica and C. ruderalis. Examples of cannabis varieties include, but are not limited to, those listed in Tables 5 and 14 available from MedReleaf Corp., Markham, Ontario, Canada.

As used herein, “plant material” includes cannabis flowers or buds as well as cannabis leaves and stems that contain detectable levels of cannabinoids. In one embodiment, the cannabis plant material comprises bracts and/or trichomes. Optionally, the cannabis plant material is processed to isolate cannabis flowers or buds from other plant material. In one embodiment, the plant material is dried plant material. In one embodiment, the dried plant material has a water content between about 6% and 12%.

As used herein “extracts” or “extract” refers to material that has been obtained from treating or processing cannabis plant material to purify and/or concentrate one or more cannabinoids produced by the cannabis plant. Examples of cannabis extracts include, but are not limited to, kief, hash, oil such as butane hash oil or supercritical CO₂ oil, rosin, tinctures etc. In one embodiment, the extract is produced by contacting cannabis plant material with a solvent. In one embodiment, the extract is cannabis oil.

As used herein “preparations” or “preparation” refers to any product made using cannabis plant material or extract thereof intended for human consumption or use. Examples of preparations include, but are not limited to, edibles, infusions, formulations, creams and capsules. In one embodiment, the preparation is a pharmaceutical composition optionally comprising one or more excipients, diluents and/or flavoring agents. In one embodiment, the preparation is a capsule containing a pre-determined amount of cannabis oil.

As used herein, “sample” refers to any biological fluid, cell or tissue sample from a subject that can be assayed for the presence or absence of genetic variants. In one embodiment, the sample comprises a nucleic acid (e.g. DNA and/or RNA). In one embodiment, the sample is a cheek swab. In one embodiment, the sample is a blood sample. Optionally, the sample may be fractionated (e.g. by centrifugation or using a column for size exclusion), concentrated or otherwise processed prior to testing the sample for genetic variants as described herein.

In one embodiment, the methods described herein further comprise obtaining a sample from a subject. In one embodiment the sample is obtained using a cheek swab or buccal swab kit suitable for collecting DNA. Kits suitable for collecting DNA are well known in the art and may include a sterile swab head and collection tube and optionally reagents to stabilize the DNA on the swab during transportation and storage, and for the efficient extraction of DNA from the swab. Optionally, the sample may be obtained by a health professional in a clinical setting. In another embodiment, the sample may be obtained at home and then sent to a laboratory or clinic for further processing such as DNA extraction and genotyping.

As used herein “dosage” refers to an amount of cannabis for administration to a subject that may be repeated over time. A dosage may include one or more doses. Optionally, the term “dosage” may refer to a discrete value or to a range such as, for example a dosage of between 0.5 grams and 1.5 grams per day.

As used herein “dose” refers to an amount of cannabis for administration to a subject within a set period of time. For example a dose of cannabis may be an amount of cannabis for a single administration or an amount of cannabis to be administered per day e.g. a maximum daily dose. In one embodiment, a dose is defined by mass, such as grams of cannabis per day. In one embodiment, the dose is defined by an amount of a specific cannabinoid, such as mg of THC and/or CBD per day. Optionally, a recommended dose is expressed as a function of body weight, such as a daily dose of 5 mg of THC per kg of body weight. Optionally, the term “dose” may refer to a discrete value or to a range such as between 0.5 grams and 1.5 grams per day or greater than 2.0 grams per day.

In one embodiment, a dosage or dose may be for a specific route of administration such as by inhalation, oral ingestion or topical administration. In one embodiment, a dosage or dose may be for a specific form of cannabis such as plant material (e.g. cannabis bud), a cannabis extract such as cannabis oil, or a cannabis preparation such as cannabis edibles or capsules. In one embodiment, a dosage or dose may be for cannabis or a cannabis product (such as an extract or preparation) with a pre-determined concentration of THC and/or CBD. For example, in one embodiment the methods described herein determine a recommended dosage of cannabis comprising a low, medium, high or extra-high dose of THC and/or CBD.

In one embodiment, the recommended dosage of cannabis is a starting dose. Subjects who have repeatedly consumed cannabis may develop an increased tolerance for cannabis and feel comfortable increasing the recommended starting dose determined using the methods described herein.

As used herein, “administration” refers to the consumption of cannabis by a subject by any route and includes self-administration. For example cannabis may be inhaled, ingested or topically applied to the skin of a subject. Various methods for inhaling cannabis include smoking cannabis rolled in a cigarette or joint, smoking cannabis in a pipe. Cannabis may also be inhaled using a vaporizer or other device designed to vaporize the active ingredients while avoiding or limiting combustion or pyrolysis. Cannabis can also be ingested alone, in the form of edibles or as a composition such as a pharmaceutical composition e.g. a pill or capsule.

As used herein, the term “subject” refers to any member of the animal kingdom, and includes mammals such as humans. The term also includes subjects such as humans who consume or are considering consuming recreational and/medicinal cannabis.

As used herein, “sensitivity” refers to the relative physiological effect of the administration of cannabinoids to a subject based on genetic factors such as the presence or absence of genetic variants. In one embodiment, the genetic factors are genetic variants associated with differences in the metabolism of cannabinoids relative to wild-type.

As used herein, “genetic variants” refers to mutated and wild-type forms of a gene sequence or other nucleic acid sequence associated with gene expression or function. Examples of genetic variants include substitutions, nonsense mutations, non-synonymous mutations, insertions and deletions. In one embodiment, the mutation is a single nucleotide polymorphism (SNP) or a coding Single Nucleotide Variant (cSNV). In one embodiment, the mutation is a copy number variant (CNV) or a loss of heterozygozity (LOH).

In one embodiment, the genetic variants are in genes associated with the physiological response of a subject to cannabis. For example, in one embodiment, the genetic variants are in genes associated with cannabinoid metabolism. Tables 1, 2 and 3 provide a list of genetic variants that modify the expression and/or activity of enzymes associated with cannabinoid metabolism.

TABLE 1
Genetic variants that modify the expression and/or activity of CYP2C19.

| | | | | | Predicted Effect |
|---|---|---|---|---|---|
| | | | | | on Cannabinoid |
| Gene | RS ID | Star Allele | Variant | Effect on gene/protein | Metabolism |
| CYP2C19 | n/a | *1 | Wild-type | — | Normal |
| CYP2C19 | rs4244285 | *2 | c.681G > A | p.Pro227=(spl?) | Deleterious |
| CYP2C19 | rs4986893 | *3 | c.636G > A | p.Trp212Ter | Deleterious |
| CYP2C19 | rs28399504 | *4 | c.1A > G | p.Met1Leu | Deleterious |
| CYP2C19 | rs56337013 | *5 | c.1297C > T | p.Arg433Trp | Deleterious |
| CYP2C19 | rs72552267 | *6 | c.395G > A | p.Arg132Gln | Deleterious |
| CYP2C19 | rs72558186 | *7 | c.819 + 2T > A | Splicing defect | Deleterious |
| CYP2C19 | rs41291556 | *8 | c.358T > C | p.Trp120Arg | Deleterious |
| CYP2C19 | rs17884712 | *9 | c.431G > A | p.Arg144His | Decreased |
| CYP2C19 | rs6413438 | *10 | c.680C > T | p.Pro227Leu | Decreased |
| CYP2C19 | rs192154563 | *16 | c.1324C > T | p.Arg442Cys | Decreased |
| CYP2C19 | rs12248560 | *17 | c.−806C > T | Promoter | Increased |
| CYP2C19 | Deleterious | *19 | c.151A > G | p.Sers1Gly | Decreased |
| CYP2C19 | rs140278421 | *22 | c.557G > C | p.Arg186Pro | Deleterious |
| CYP2C19 | rs118203757 | *24 | c.1004G > A | p.Arg335Gln | Deleterious |
| CYP2C19 | rs118203759 | *25 | c.1344C > G | p.Phe448Leu | Decreased |
| CYP2C19 | n/a | *26 | c.766G > A | p.Asp256Asn | Decreased |
| CYP2C19 | rs12769205 | *35 | c.33223A > G | Splicing defect | Deleterious |

TABLE 2
Genetic variants that modify the expression and/or activity of CYP2C9

| | | | | | Predicted Effect |
|---|---|---|---|---|---|
| | | | | | on Cannabinoid |
| Gene | RS ID | Star Allele | Variant | Effect on gene/protein | Metabolism |
| CYP2C9 | n/a | *1 | Wild-type | — | Normal |
| CYP2C9 | rs1799853 | *2 | c.430C > T | p.Arg144Cys | Deleterious |
| CYP2C9 | rs1057910 | *3 | c.1075A > C | p.lle359Leu | Deleterious |
| CYP2C9 | rs56165452 | *4 | c.1076T > C | p.lle359Thr | Decreased |
| CYP2C9 | rs28371686 | *5 | c.1080C > G | p.Asp360Glu | Decreased |
| CYP2C9 | rs9332131 | *6 | c.818delA | p.Lys273Argfs | Deleterious |
| CYP2C9 | rs7900194 | *8 | c.449G > A | p.Arg150His | Decreased |
| CYP2C9 | rs28371685 | *11 | c.1003C > T | p.Arg335Trp | Decreased |
| CYP2C9 | rs9332239 | *12 | c.1465C > T | p.Pro489Ser | Decreased |
| CYP2C9 | rs72558187 | *13 | c.269T > C | p.Leu90Pro | Decreased |
| CYP2C9 | rs72558190 | *15 | c.485C > A | p.Ser162Ter | Deleterious |
| CYP2C9 | rs72558188 | *25 | c.353_362del- | | Deleterious |
| | | | AGAAATGGAA | | |
| CYP2C9 | rs57505750 | *31 | c.980T > C | p.lle327Thr | Decreased |

TABLE 3
Genetic variants that modify the expression and/or activity of CYP3A4.

| | | | | | Predicted Effect |
|---|---|---|---|---|---|
| | | | | | on Cannabinoid |
| Gene | RS ID | Star Allele | Variant | Effect on gene/protein | Metabolism |
| CYP3A4 | n/a | *1 | Wild-type | — | |
| CYP3A4 | rs55785340 | *2 | c.664T > C | p.Ser222Pro | Decreased |
| CYP3A4 | rs72552799 | *8 | c.389G > A | p.Arg130Gln | Decreased |
| CYP3A4 | rs67784355 | *11 | c.1088C > T | p.Thr363Met | Decreased |
| CYP3A4 | rs12721629 | *12 | c.1117C > T | p.Leu373Phe | Decreased |
| CYP3A4 | rs4986909 | *13 | c.1247C > T | p.Pro416Leu | Decreased |
| CYP3A4 | rs12721627 | *16 | c.554C > G | p.Thr185Ser | Decreased |
| CYP3A4 | rs4987161 | *17 | c.566T > C | p.Phe189Ser | Decreased |
| CYP3A4 | rs67666821 | *20 | c.1461_1462insA | p.Pro488Thrfs | Deleterious |
| CYP3A4 | rs35599367 | *22 | c.522191C > T | Splicing defect | Decreased |
| CYP3A4 | rs138105638 | *26 | c.802C > T | p.Arg268Ter | Deleterious |

In one embodiment, the genetic variant modifies the expression and/or activity of a cytochrome P450 enzyme associated with cannabinoid metabolism such that there is a corresponding modification of the metabolism of the cannabinoid in vivo in a subject carrying the variant relative to wild type. As used herein a “deleterious” variant is a variant that is predicted to abolish the expression and/or activity of the gene encoded by the deleterious variant. As used herein, a “decreased” variant is a variant that is predicted to decrease the expression and/or activity of the gene encoded by the deleterious variant relative to wild-type. In one embodiment, a “decreased” gene variant exhibits detectable expression and/or activity of a gene product that is decreased relative to the expression and/or activity of the wild-type gene product. As used herein an “increased” variant is a variant that is predicted to increase the expression and/or activity of the gene encoded by the increased variant relative to wild-type.

Tables 15 and 16 identify SNP variants associated with responding to treatment for pain with cannabis containing CBD (Table 15) or cannabis containing THC (Table 16).

In one embodiment, the method comprises determining the presence or absence of one or more genetic variants in or near a gene selected from CYP2C9, CYP3A4 and CYP2C19. In one embodiment, the genetic variant is a variant listed in Tables 1, 2, 3, 15 or 16. In one embodiment, the genetic variant is a variant associated with CBD metabolism, optionally a variant in CYP2C19 or CYP3A4 listed in Tables 1 or 3. In one embodiment, the genetic variant is a variant associated with THC metabolism, optionally a variant in CYP2C9 or CYP3A4 listed in Table 2 or 3. In some embodiments, the methods described herein include determining the presence or absence of genetic variants associated with THC metabolism and the presence or absence of genetic variants associated with CBD metabolism.

For example, in one embodiment, the variant is a “deleterious” variant such as CYP2C19*2 that results in an aberrant splice site and the production of a truncated and non-functioning protein. In one embodiment, a subject with one or two deleterious alleles exhibits a relative decrease in CBD metabolism relative to wild-type. In one embodiment the variant is a “decreased” variant such as CYP3A4*8 that results in a missense mutation (p.Arg130Gln) and a protein with decreased activity relative to wild-type. In one embodiment, a subject with one or two decreased alleles exhibits a relative decrease in cannabinoid metabolism relative to wild-type. In one embodiment, the variant is an “increased” variant such as CYP2C19*17 that results in the increased transcription of CYP2C19 compared to wild-type. In one embodiment, subjects with one or two increased alleles exhibits a relative increase in cannabinoid metabolism relative to wild-type. A skilled person will appreciate that the presence of “deleterious” and “decreased” variants will generally result in a subject with a higher sensitivity to cannabinoids and a lower recommended dose relative to wild-type subjects. The presence of an “increased” variant will generally results in a subject with a lower sensitivity to cannabinoids and a higher recommended dose relative to wild-type subjects.

In one embodiment, a subject with wild-type alleles for the CYP2C19, CYP3A4 and CYP2C9 variants described herein is expected to metabolize cannabinoids such as THC and CBD relatively rapidly and may be recommended a dosage of “high” and/or identified as having a normal sensitivity to cannabis. In one embodiment, a cannabis compatibility score is generated as set out below based on the presence or absence of a plurality of variants for each allele.

In one embodiment, the method comprises determining the presence or absence of a subset of genetic variants listed in Tables 1, 2, 3, 15 or 16. For example, in one embodiment, the method comprises determining the presence or absence of one or more of the genetic variants listed in Table 10. In one embodiment, the method comprises determining the presence of absence of 2, 3, 4, 5, 6, 7, 8, 9, 10, 11, 12, 13, 14 or 15 of the genetic variants listed in Table 10. In one embodiment, determining the presence or absence of one or more of the genetic variants listed in Table 10 is expected to be particularly useful for determining a recommended dose of cannabis for a subject and/or determining the sensitivity of a subject to cannabis.

In one embodiment, determining the presence or absence of one or more genetic variants in the sample from the subject comprises testing the sample for the one or more genetic variants. Different techniques known in the art for genotyping may be used in the methods described herein to identify genetic variants in a sample. For example, suitable techniques for detecting a genetic variant as described herein include, but are not limited to, nucleic acid sequencing methods, hybridization with probes specific for a particular nucleic acid sequence, primer extension reactions, PCR based methods (optionally multiplex PCR), or the use of expression arrays such as microarrays and the like.

Optionally, in one embodiment the method comprises detecting the presence or absence of a coding genetic variant by detecting the presence or absence of a variant polypeptide corresponding to the genetic variant as described herein. Suitable methods for detecting a variant polypeptide include, but are not limited to, the use of mass spectroscopy, Western blots, enzyme-linked immunosorbant assays (ELISA), and immunoprecipitation followed by sodium-dodecyl sulfate polyacrylamide gel electrophoresis (SDS-PAGE), immunocytochemistry and protein sequencing methods. In one embodiment, the method comprises contacting the sample with a binding protein or antibody that selectively binds a variant polypeptide corresponding to a genetic variant as described herein.

In one embodiment, the methods described herein include generating a cannabis compatibility score for a subject based on the presence or absence of one or more genetic variants. In one embodiment, the cannabis compatibility is compared to one or more control scores, wherein each control score is associated with a recommended dosage of cannabis.

The cannabis compatibility score may be based on the determination of genetic variants that are associated with the physiological response of a subject to one or more cannabinoids. For example, in one embodiment the cannabis compatibility score is a THC compatibility score based on the presence or absence of one or more genetic variants in CYP2C9 and/or CYP3A4 associated with the metabolism of THC in vivo. In another embodiment the cannabis compatibility score is a CBD compatibility score based on the presence or absence of one or more genetic variants in CYP2C19 and/or CYP3A4 associated with the metabolism of CBD in vivo. In one embodiment, the cannabis compatibility score is based on the presence or absence of genetic variants associated with the metabolism of THC and CBD in vivo, optionally one or more genetic variants in genes selected from CYP2C9, CYP3A4 and CYP2C19. In one embodiment, the cannabis compatibility score is based on the presence or absence of one or more genetic variants listed in Table 1, 2 and/or 3 or the genetic variants listed in Table 10. Optionally, a cannabis compatibility score is generated for a subject identified as responsive to treating paint with CBD or THC based on the presence or absence of the SNP variants listed in Table 15 or 16.

In one embodiment, the cannabis compatibility score is indicative of the genetic sensitivity of the subject to cannabis or to cannabinoids. For example, in one embodiment a higher cannabis compatibility score is indicative of relatively high or fast metabolism of cannabis relative to a lower cannabis compatibility score that is indicative of relatively slow or poor metabolism of cannabis. The cannabis compatibility score may be compared to one or more control scores in order to determine a recommended dosage of cannabis and/or a recommended dosage of specific cannabinoid such as THC or CBD.

Example 2 provides an exemplary embodiment for generating cannabis compatibility scores for THC metabolism and/or CBD metabolism based on the genetic variants identified in Table 10. In one embodiment, a THC and/or CBD compatibility score is determined based on the scoring matrix and genotypes for a subject as set forth in Table 11. Optionally, a specific variety of cannabis is selected for the treatment of a specific medical condition as set out in Table 13. A recommended dosage, optionally a recommended dosage of THC and/or CBD or a recommended dosage for a specific variety of cannabis, may then be determined by reference to the control scores and dosages in Tables 11 and 12. Varieties of cannabis such as Avidekel^{MR}, Cognitiva^{MR}, Eran Almog^{MR}, Luminarium^{MR}, Midnight^{MR}, Nollia^{MR} and Sedamen^{MR} are available from MedReleaf Corp, Markham, ON, Canada).

In one embodiment, the recommended dosage of cannabis is a low dose, a medium dose or a high dose and the low dose is less than 343 mg of THC per day, the medium dose is between 343 mg and 573 mg of THC per day, and the high dose is greater than 573 mg of THC per day. Optionally, in one embodiment the recommended dosage of cannabis is a low daily dose of less than 5.72 mg/kg, a medium daily dose of between 5.72 mg/kg and 9.55 mg/kg, or a high daily dose of greater than 9.55 mg/kg.

In one embodiment, the recommended dosage of cannabis is a low dose, a medium dose, a high dose or an extra-high dose and the low dose comprises less than 195 mg of CBD per day, the medium dose is between 195 mg and 325 mg of CB per day, the high dose is greater than 325 mg and less than or equal to 400 mg of CBD per day, and the extra-high dose is greater than 400 mg of CBD per day. Optionally, in one embodiment the recommended dosage of cannabis is a low daily dose of CBD of less than 3.25 mg/kg, a medium daily dose of CBD between 3.25 mg/kg and 5.42 mg/kg, a high daily dose of CBD greater than 5.42 mg/kg and less than or equal to 6.67 mg/kg or an extra-high daily dose of CBD greater than 6.67 mg/kg.

In one embodiment, recommended dosage of cannabis may depend on the intended route of administration and/or the form of cannabis, for example whether the cannabis is cannabis plant material such as cannabis buds intended for vaporization or a cannabis extract such as cannabis oil and the relative concentration of cannabinoids in the cannabis.

In one embodiment, the methods described herein are useful for determining a recommended variety and/or dosage of cannabis for the treatment of a medical condition in a subject in need thereof. As set out in Example 1, clinical survey data of a population of patients who had been prescribed medical marijuana was used to identify specific varieties that were particularly efficacious for the treatment of specific medical conditions such as anxiety, appetite disorder, depression, inflammation, pain, nausea and/or vomiting, seizures, or sleep disorder. The methods described herein may therefore be used to provide a recommended dosage and variety for the treatment of specific medical condition. As set out in Examples 3 and 4, SNP variants have been identified that are associated with the efficacious treatment of pain using cannabis comprising CBD or THC.

In one embodiment, the one or more SNP variants are selected rs734969, exm2271332, kgp10780705, kgp12541436, kgp8466518, kgp9307988 and rs10518253. In one embodiment, the presence of an A allele at rs734969, a T allele at exm2271332, a T allele at kgp10780705, an A allele at kgp12541436, an A allele at kgp8466518, a T allele at kgp9307988 and/or a A allele rs10518253 is associated with a good response to the treatment of pain with CBD, optionally with Avidekel.

In one embodiment, the one or more SNP variants are selected from rs1856908, kgp10378560, kgp10780705, kgp8413369, rs10786189, rs1856908, rs28670611, rs7255901 and rs7919934. In one embodiment, the presence of an T allele at rs1856908, an A allele at kgp10378560, a T allele at kgp10780705, a T allele at kgp8413369, an A allele at rs10786189, a T allele at rs1856908, an A allele at rs28670611, an A allele at rs7255901 and/or an A allele at rs7919934 is associated with a good response to the treatment of pain with THC, optionally with Sedamen or Luminarium.

As used herein, “treating” or “treatment” of a medical condition refers to an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. In one embodiment, treatment of a medical condition comprises the administration to a subject or use of a therapeutically effective amount of one or more cannabinoids as described herein and optionally consists of a single administration, or alternatively comprises a series of administrations. In one embodiment, the treatment of a medical condition comprises the use or administration of a recommended dosage and/or variety of cannabis as described herein.

A number of different medical conditions are known in the art to benefit from treatment with cannabis, optionally with THC and/or CBD. Accordingly, in one embodiment the methods described herein are useful for determining a recommended dosage and/or variety of cannabis for a medical condition such as for the relief of pain and treatment in palliative care, for reducing stress and fatigue, for mood enhancement, for relief from nausea, vomiting, headaches, muscle spasms, seizures, wasting syndrome and loss of appetite in AIDS and cancer patients, for maintaining general health and wellbeing, for treating symptoms associated with anorexia nervosa, Crohn's disease, multiple sclerosis, amyotrophic lateral sclerosis, fibromyalgia, spinal cord injury, epilepsy, migraines, arthritides and musculoskeletal disorders, neuromuscular diseases and disorders, such as, but not limited to, cerebrovascular accident (stroke), Parkinson's disease, multiple sclerosis, rheumatism, myasthenia gravis, Huntington's disease, amyotrophic lateral sclerosis, epilepsy, convulsant and movement disorders, cervical dystonia, Meige's syndrome, glaucoma, asthma, hypertension, mental illness such as, but not limited to, anxiety, depression, sleep disorders, post-traumatic stress disorder, alcohol and opioid withdrawal symptoms, schizophrenia and psychosis, attention and hyperactivity disorders, Alzheimer's disease, and dementia. In one embodiment, the medical condition is a skin infection and/or irritation, such as, but not limited to, bacterial skin infections, dermatitis, atrophie blanche, eczema, burns, rashes from skin allergies and reactions, psoriasis, pruritis and neuropathic pain. In one embodiment, the medical condition is an inflammatory disease such as, but not limited to inflammatory skin diseases. In one embodiment, the medical condition is a gastrointestinal system disorder, irritable bowel syndrome, or inflammatory bowel diseases. In one embodiment, the medical condition is a disease of the liver, Wilson's disease, metabolic syndrome, obesity, diabetes, or a disease of the pancreas. In one embodiment, the medical condition is atherosclerosis, osteoarthritis, or osteoporosis. In one embodiment, the medical condition is a microbial infection, optionally a viral or bacterial infection. In one embodiment, the medical condition is intraocular pressure. In one embodiment, the medical condition is cancer.

In one embodiment, the medical condition is anxiety, appetite disorder, depression, inflammation, pain, nausea and/or vomiting, seizures, or a sleep disorder. In one embodiment, the medical condition includes symptoms associated with PTSD. In one embodiment, the methods described herein involve recommending a dosage of a specific variety of cannabis based on a desired therapeutic benefit, specifically the treatment of a medical condition. As set out in the Examples certain varieties of cannabis have been demonstrated to be particularly efficacious for the treatment of specific medical conditions.

In one embodiment, the recommended variety of cannabis is a variety available from MedReleaf Corp, Markham, ON, Canada. Varieties of cannabis available from MedReleaf Corp, include but are not limited to, Avidekel^{MR}, Cognitiva^{MR}, Eran Almog^{MR}, Luminarium^{MR}, Midnight^{MR}, Nollia^{MR} and Sedamen^{MR}.

In one embodiment, the medical condition is anxiety and the recommended variety is Midnight^{MR}, Avidekel^{MR} or Luminarium^{MR}.

In one embodiment, the medical condition is an appetite disorder and the recommended variety is Cognitiva^{MR}, Sedamen^{MR} or Eran Almog^{MR}. In one embodiment, the medical condition is depression and the recommended variety is Midnight^{MR}, Luminarium^{MR} or Avidekel^{MR}.

In one embodiment, the medical condition is inflammation and the recommended variety is Avidekel^{MR}, Midnight^{MR} or Nollia^{MR}.

In one embodiment, the medical condition is pain and the recommended variety is Midnight^{MR}, Luminarium^{MR}, Avidekel^{MR} or Sedamen^{MR}.

In one embodiment, the medical condition is nausea and/or vomiting and the recommended variety is Midnight^{MR}, Sedamen^{MR} or Avidekel^{MR}. In one embodiment, the medical condition is seizures and the recommended variety is Avidekel^{MR} or Midnight^{MR}.

In one embodiment, the medical condition is a sleep disorder, and the recommended variety is Midnight^{MR}, Sedamen^{MR} or Eran Almog^{MR}.

The above disclosure generally describes the present invention. A more complete understanding can be obtained by reference to the following specific examples of certain embodiments of the invention.

All publications, biological sequences or sequence identifiers, patents and patent applications are herein incorporated by reference in their entirety to the same extent as if each individual publication, biological sequence, patent or patent application was specifically and individually indicated to be incorporated by reference in its entirety.

### Example 1: Efficacy of Different Varieties of Medical Cannabis for the Treatment of Medical Conditions and Symptoms

Traditionally, cultivars of Cannabis sativa L. have been divided into sub-species based on their morphological properties, metabolic profile, and geographical origin. Interbreeding subspecies renders hybrids characterised by varying sativa and indica profiles, and unique cannabinoid ratios. As cannabinoid compounds like tetrahydrocannabinol (THC) and cannabidiol (CBD) are thought to be primarily responsible for the physiological effects of cannabis, unique varietal profiles may provide different therapeutic benefits suitable for managing different symptoms and conditions. A study was performed to assess the efficacy of different cannabis varieties in patients using medical cannabis from one Canadian licensed provider (MedReleaf Corp). Information pertaining to current medical conditions, symptoms, and quality of life were collected through a voluntary online survey administered to patients after registration, and at 4 and 10 month follow-up intervals. 837 patients provided information about their experience with medical cannabis at 4-month follow-up. Patients reported that the variety Midnight^{MR} (sativa-leaning, 8-11% THC, 11-14% CBD) was most efficacious for reducing pain (27.4%), and that Luminarium^{MR} (very sativa-dominant, 25-28% THC, 0% CBD) was effective for managing both anxiety disorder (30.4%) and depression (35.5%). Patients most commonly attributed improvements in sleep (29.0%), appetite (24.8%), and bowel function (24.6%) to Midnight^{MR}, improvements in concentration (22.0%) to Cognitiva^{MR} (sativa-leaning, 15-18% THC, 0% CBD), and improvements in sexual function (26.5%) to Luminarium^{MR}. The efficacy of different cannabis varieties in managing various symptoms should be further investigated in a controlled clinical setting, to enable patients and physicians to make informed decisions on which varieties are best suited to achieve optimal symptom management.

### Methods

### Survey Design

An online survey was designed in consultation with physicians and nurses experienced in prescribing medical cannabis, and with reference to relevant scientific literature. Existing validated questionnaires that assessed pain and quality of life were adapted to assess specific attributes of the target patient population. Questions assessing patients' pain levels were measured on a scale of 0-10 (with 0 being no pain and 10 being worst possible pain), and patients' ability to cope with their pain was assessed based on an adaption of the Pain Self-Efficacy Questionnaire (Nicholas, 2007). Quality of life (QOL) questions were based on two commonly used and validated methods of QOL assessment (Burckhardt and Anderson, 2003; Fletcher et al., 1992). The survey was dynamic, and patients were given questions based on relevance as determined by their answers to earlier questions (e.g. patients were not asked about their pain experience if they did not indicate pain as a symptom or condition). Since the survey was customized to best assess each patient's unique characteristics, not all questions were mandatory and not all patients answered every question.

### Baseline

Patients were invited to complete an intake survey at the time of registration, which is considered “baseline”. The survey collected demographic information and information pertaining to current medical conditions and symptoms. Condition and symptom severity was reported as mild, moderate, or severe. Patients were asked additional detailed questions relevant to the symptoms or conditions they selected. QOL was assessed by asking patients to report on their experiences with items such as sleep, appetite, concentration, bowel activity, and sexual function, by selecting from the options “severe difficulty”, “moderate difficulty”, “no difficulty”, “good”, and “very good”.

### Follow-Up

Patients were invited to complete a follow-up survey 4 and 10 months following completion of the initial intake survey. Patients were asked to report any changes they had experienced to their symptoms, conditions, or QOL. They were also asked about their experience with medical cannabis, and if they had experienced any changes in their symptoms or conditions, which of the cannabis varieties they perceived the changes could be attributed to. Cannabis varieties were categorized based on their approximate sativa and indica character. Sativa-leaning strains consist of 50-60% sativa character, sativa-dominant strains consist of 61-70% sativa character, and very sativa-dominant strains consist of >70% sativa character. Indica strains are similarly characterised.

### Data Analysis

Baseline survey data was filtered to include only patients who indicated which strains they took at the 4-month follow-up for analysis.

### Results

### Baseline Characteristics

In total, 837 patients reported which cannabis varieties they consumed at 4-month follow-up. The demographic and lifestyle characteristics of these patients at baseline are presented in Table 4. The majority of patients were male (68.8%), Caucasian (83.4%), and reported that they have prior experience with cannabis at the time of survey completion (78.9%). The average age of patients was 44.9 years old, with an age range from one year to 80 years old. Most patients belonged in the age bracket of 50-59 (28.7%). The most common medical conditions reported in this patient cohort at baseline included depression (34.5%), anxiety disorder (34.3%), post-traumatic stress disorder (PTSD, 25.9%), and sleep disorder (25.7%). Pain was reported by 33.0% of patients at baseline. The most common symptoms at baseline included anxiety (40.7%), sleep problems (38.4%), depression (29.9%), insomnia (29.3%), and headache (20.4%).

Properties of Cannabis Varieties to which Improvement was Most Often Attributed

651 patients reported on which cannabis varieties they felt contributed most to any improvements in overall health. These are presented in Table 5, along with information pertaining to each variety's composition, THC and CBD content. The variety Midnight^{MR} (sativa-leaning, 8-11% THC, 11-14% CBD) was the most popular strain overall, with 18.7% of all patients perceiving it to be the most beneficial cannabis strain contributing to improvements in overall health. This was followed by Avidekel^{MR} (indica-leaning, 0.1-0.8% THC, 15-18% CBD) preferred by 13.7% of patients, and Sedamen^{MR} (indica-dominant, 21-24% THC, 0% CBD) preferred by 9.4% of patients.

### Improvements in Conditions, Symptoms, and QOL Items

Table 6 lists the top 5 conditions reported by patients, and the three cannabis varieties to which improvements in conditions at 4-month follow up were attributed. These conditions include anxiety, depression, sleep disorder, arthritis, and post-traumatic stress disorder. Improvement was most commonly reported by patients experiencing anxiety as a condition at intake. Out of 69 patients, 35.5% attributed their anxiety reduction to the variety Luminarium^{MR} (very sativa-dominant, 25-28% THC, 0% CBD). This was followed by Midnight^{MR} (27.5%) and Avidekel^{MR} (21.7%).

Six of the most common symptoms for which patients reported improvements after 4 months of medical cannabis use include anxiety (as a symptom rather than condition), sleep problems, depression, insomnia, headaches, and exhaustion (Table 7). 27.3% of 341 patients attributed improvements in their symptom of anxiety to Sedamen^{MR}. This was followed by Luminarium^{MR} (25.2%), and Cognitiva^{MR} (22.0%). 27.7% out of 321 patients attributed improvements in sleep problems to Luminarium^{MR}, followed by Midnight^{MR} (24.6%), and Avidekel^{MR} (20.2%).

At the 4-month follow-up, patients were asked about which cannabis varieties they felt were responsible for changes, if any, in five quality of life measures, including sleep, appetite, concentration, bowel activity, and sexual function (Table 8). 224 and 175 patients attributed improvements in sleep and bowel function to three specific strains: Midnight^{MR} (29.0% and 24.6%, respectively), Sedamen^{MR} (22.8% and 21.1%, respectively), and Avidekel^{MR} (16.5% and 20.6%, respectively). Improvements in appetite, with 302 total responses, were attributed to Midnight^{MR} (24.8%), Cognitiva^{MR} (24.2%), and Sedamen^{MR} (22.8%). For improvements in concentration, responses from 191 patients indicate that Cognitiva^{MR} (22.0%), Midnight^{MR} (20.4%), and Avidekel^{MR} (19.4%) were perceived to be the most beneficial strains.

### Improvements in Pain as a Symptom at 4 Months and 10 Months

At 4 months, 259 patients reported on the cannabis varieties that they perceived had caused the greatest reduction in pain levels. A majority of these patients identified Midnight^{MR} (27.4%) as the most helpful, followed by Sedamen^{MR} (22.8%), and Avidekel^{MR} (22.4%). Of the 138 patients who also reported improvement in pain in the 10-month follow-up, most attributed their pain reduction to the variety Sedamen^{MR} (29.0%), followed by Luminarium^{MR} (26.1%) and Midnight^{MR} (26.1%).

### Discussion

Different medical cannabis varieties were found to have varying efficacies for the management of different symptoms or conditions. For example, Eran Almog^{MR} (very indica-dominant, 25-28% THC, 0% CBD) was the strain most beneficial for insomnia and sleep disorder, but was the ninth most popular strain overall, and did not appear in the top three varieties for the management of any other condition, symptom, or QOL measure. Similarly, Stellio^{MR} was identified as the most beneficial for PTSD and exhaustion, but was reported to be the 14^{th} most effective for overall health, and did not appear to be associated with any other common conditions or symptoms.

On the other hand, several varieties such as Avidekel^{MR} and Midnight^{MR} were found to be effective across a range of conditions and symptoms. Both varieties contain high CBD content (Midnight^{MR} 11-14% CBD, Avidekel^{MR} 15-18% CBD), with Midnight^{MR} also containing moderate levels of THC (Midnight^{MR} 8-11% THC, Avidekel^{MR} 0.1-0.8% THC). These two varieties often appeared together as two of the top three strains perceived to be most effective for multiple indications including anxiety disorder, arthritis, sleep problems, and headache. Moreover, Midnight^{MR} and Avidekel^{MR} both appeared as the top strains for improvements in three out of five QOL items including sleep, concentration, and bowel activity. In these QOL categories, Midnight^{MR} was always found to be preferred over Avidekel^{MR}. This suggests that a combination of CBD and THC may be more clinically useful than THC or CBD on their own.

Reported improvements in pain 4 and 10 months after intake was remarkably consistent, with the varieties Midnight^{MR}, Sedamen^{MR}, Avidekel^{MR}, and Luminarium^{MR} identified as the top four strains by patients at both follow-up intervals (Table 9). This suggests that these strains may be optimal for pain management. In particular, Midnight^{MR} and Avidekel^{MR} are likely to contribute to pain reduction in this population due to the anti-inflammatory effects associated with CBD (Zhornitsky and Potvin, 2012).

TABLE 4
Baseline demographics of patients who indicated cannabis
varieties associated with symptom improvement
PTSD: Post-traumatic stress disorder

| | |
|---|---|
| Demographic | n (%) |

Gender (Total n = 837)

| | |
|---|---|
| Male | 575 (68.8%) |
| Female | 259 (30.9%) |
| Other | 3 (0.4%) |

Ethnicity (Total n = 836)

| | |
|---|---|
| Caucasian | 697 (83.4%) |
| Spanish/Hispanic/Latino | 4 (0.5%) |
| Native Canadian | 40 (4.8%) |
| Black/African American | 8 (1.0%) |
| Asian | 10 (1.2%) |
| Pacific Islander | 1 (0.1%) |
| Prefer not to answer | 31 (3.7%) |
| Other | 45 (5.4%) |

Age in years (Total n = 833)

| | |
|---|---|
| 0-19 | 22 (2.6%) |
| 19-29 | 66 (7.9%) |
| 30-39 | 209 (25.1%) |
| 40-49 | 179 (21.5%) |
| 50-59 | 239 (28.7%) |
| 60-69 | 96 (11.5%) |
| 70 | 22 (2.6%) |
| Average (min, max) | 44.9 (1, 80) |

Other conditions (Total n = 837)

| | |
|---|---|
| Depression | 289 (34.5%) |
| Anxiety disorder | 287 (34.3%) |
| PTSD | 217 (25.9%) |
| Sleep disorder | 215 (25.7%) |

Previous experience with cannabis (Total n = 835)

| | |
|---|---|
| Yes | 659 (78.9%) |
| No | 118 (14.1%) |
| Prefer not to answer | 58 (6.9%) |

TABLE 5
Properties of 15 most popular cannabis varieties perceived to be
most beneficial overall

| | Patients | | | |
|---|---|---|---|---|
| Variety | found most beneficial n (%) | Composition | % THC | % CBD |
| Midnight^{MR} | 122 (18.7%) | sativa-leaning | 8-11% | 11-14% |
| Avidekel^{MR} | 89 (13.7%) | indica-leaning | 0.1-0.8% | 15-18% |
| Sedamen^{MR} | 61 (9.4%) | indica-dominant | 21-24% | 0 |
| Luminarium^{MR} | 57 (8.8%) | sativa-dominant | 25-28% | 0 |
| Cognitiva^{MR} | 51 (7.8%) | sativa-leaning | 15-18% | 0 |
| Remissio^{MR} | 29 (4.5%) | indica-dominant | 24-27% | 0 |
| Alaska^{MR} | 26 (4.0%) | sativa-dominant | 20-23% | 0 |
| Erez^{MR} | 24 (3.7%) | indica-dominant | 20-23% | 0 |
| Eran Almog^{MR} | 20 (3.1%) | very indica-dominant | 25-28% | 0 |
| Contenti^{MR} | 16 (2.5%) | indica-leaning | 15-18% | 0 |
| Cerebri^{MR} | 14 (2.2%) | very indica-dominant | 25-28% | 0 |
| Voluptas^{MR} | 14 (2.2%) | very sativa-dominant | 20-23% | 0 |
| Elevare^{MR} | 13 (2.0%) | very sativa-dominant | 24-27% | 0 |
| Stellio^{MR} | 11 (1.7%) | indica-dominant | 23-26% | 0 |
| Or^{MR} | 10 (1.5%) | indica-dominant | 20-23% | 0 |

TABLE 6
Top 3 cannabis strains associated with condition improvement PTSD:
Post-traumatic stress disorder

| Condition (Total n) | 1^{st} | 2^{nd} | 3^{rd} |
|---|---|---|---|
| Anxiety disorder (69) | Luminarium^{MR} | Midnight^{MR} | Avidekel^{MR} |
| | n = 21 (30.4%) | n = 19 (27.5%) | n = 15 (21.7%) |
| Depression (62) | Luminarium^{MR} | Avidekel^{MR} | Alaska^{MR} |
| | n = 22 (35.5%) | n = 18 (29%) | n = 16 (25.8%) |
| Sleep disorder (53) | Eran Almog^{MR} | Sedamen^{MR} | Midnight^{MR} |
| | n = 17 (32.1%) | n = 17 (32.1%) | n = 11 (20.8%) |
| Arthritis (46) | Midnight^{MR} | Avidekel^{MR} | Sedamen^{MR} |
| | n = 18 (39.1%) | n = 15 (32.6%) | n = 13 (28.3%) |
| PTSD (41) | Stellio^{MR} | Luminarium^{MR} | Sedamen^{MR} |
| | n = 15 (36.6%) | n = 13 (31.7%) | n = 12 (29.3%) |

TABLE 7
Top 3 cannabis strains associated with symptom improvement

| Symptom (Total n) | 1^{st} | 2^{nd} | 3^{rd} |
|---|---|---|---|
| Anxiety (341) | Sedamen^{MR} | Luminarium^{MR} | Cognitiva^{MR} |
| | n = 93 (27.3%) | n = 86 (25.2%) | n = 75 (22.0%) |
| Sleep problems | Luminarium^{MR} | Midnight^{MR} | Avidekel^{MR} |
| (321) | n = 89 (27.7%) | n = 79 (24.6%) | n = 65 (20.2%) |
| Depression (250) | Luminarium^{MR} | Avidekel^{MR} | Alaska^{MR} |
| | n = 80 (32%) | n = 63 (25.2%) | n = 50 (20%) |
| Insomnia (245) | Eran Almog^{MR} | Sedamen^{MR} | Midnight^{MR} |
| | n = 68 (27.8%) | n = 61 (24.9%) | n = 45 (18.4%) |
| Headache (171) | Midnight^{MR} | Avidekel^{MR} | Sedamen^{MR} |
| | n = 53 (31%) | n = 40 (23.4%) | n = 39 (22.8%) |
| Exhaustion (164) | Stellio^{MR} | Luminarium^{MR} | Sedamen^{MR} |
| | n = 43 (26.2%) | n = 38 (23.2%) | n = 29 (17.7%) |

TABLE 8
Top 3 strains associated with QOL improvement
QOL: quality of life

| Symptom (Total n) | 1^{st} | 2^{nd} | 3^{rd} |
|---|---|---|---|
| Sleep (224) | Midnight^{MR} | Sedamen^{MR} | Avidekel^{MR} |
| | n = 65 (29.0%) | n = 51 (22.8%) | n = 37 (16.5%) |
| Appetite (302) | Midnight^{MR} | Cognitiva^{MR} | Sedamen^{MR} |
| | n = 75 (24.8%) | n = 73 (24.2%) | n = 69 (22.8%) |
| Concentration (191) | Cognitiva^{MR} | Midnight^{MR} | Avidekel^{MR} |
| | n = 42 (22.0%) | n = 39 (20.4%) | n = 37 (19.4%) |
| Bowel function (175) | Midnight^{MR} | Sedamen^{MR} | Avidekel^{MR} |
| | n = 43 (24.6%) | n = 37 (21.1%) | n = 36 (20.6%) |
| Sexual function (170) | Luminarium^{MR} | Sedamen^{MR} | Cognitiva^{MR} |
| | n = 45 (26.5%) | n = 44 (25.9%) | n = 36 (21.2%) |

TABLE 9
Top 10 strains associated with pain improvement at
4-month follow-up and 10-month follow-up

| 4-months | | 10-months | |
|---|---|---|---|
| Strain | n (%) | Strain | n (%) |
| Midnight^{MR} | 71 (27.4%) | Sedamen^{MR} | 40 (29.0%) |
| Sedamen^{MR} | 59 (22.8%) | Luminarium^{MR} | 36 (26.1%) |
| Avidekel^{MR} | 58 (22.4%) | Midnight^{MR} | 36 (26.1%) |
| Luminarium^{MR} | 46 (17.8%) | Avidekel^{MR} | 34 (24.6%) |
| Cognitiva^{MR} | 45 (17.4%) | Remissio^{MR} | 22 (15.9%) |
| Eran Almog^{MR} | 39 (15.1%) | Cognitiva^{MR} | 22 (15.9%) |
| Stellio^{MR} | 35 (13.5%) | Eran Almog^{MR} | 22 (15.9%) |
| Alaska^{MR} | 30 (11.6%) | Alaska^{MR} | 21 (15.2%) |
| Bellis^{MR} | 24 (9.3%) | Cerebri^{MR} | 20 (14.5%) |
| Remissio^{MR} | 20 (7.7%) | Stellio^{MR} | 19 (13.8%) |
| Total n | 259 | Total n | 138 |

### Example 2: Cannabis Compatibility Scores for THC and CBD

The 15 variants listed in Table 10 were selected as likely to play a role in THC and CBD metabolism and be informative for determining a recommended dosage of cannabis in a genetic cannabis compatibility test. 14 of these variants are thought to decrease metabolism of cannabinoids, and 1 increases metabolism of cannabinoids.

TABLE 10
Subset of 15 genetic variants in CYP2C19, CYP2C9 and
CYP3A4 for determining cannabinoid metabolism in a
genetic cannabis compatibility test

| Gene | | | |
|---|---|---|---|
| (Involved in | Mutant | | Effect on |
| THC or CBD | Allele | Star | cannabinoid |
| metabolism) | RS ID | Allele | metabolism |
| CYP2C19 | n/a | *1 | Wildtype |
| (CBD) | rs4244285 | *2 | Deleterious |
| | rs4986893 | *3 | Deleterious |
| | rs41291556 | *8 | Deleterious |
| | rs12248560 | *17 | Increased |
| | rs12769205 | *35 | Deleterious |
| CYP2C9 | n/a | *1 | Wildtype |
| (THC) | rs1799853 | *2 | Deleterious |
| | rs1057910 | *3 | Deleterious |
| | rs28371686 | *5 | Decreased |
| | rs7900194 | *8 | Decreased |
| | rs9332239 | *12 | Decreased |
| CYP3A4 | n/a | *1 | Wildtype |
| (THC + CBD) | rs55785340 | *2 | Decreased |
| | rs72552799 | *8 | Decreased |
| | rs12721629 | *12 | Decreased |
| | rs67666821 | *20 | Deleterious |
| | rs35599367 | *22 | Decreased |

Individuals can be genotyped and assigned a score based on their genotype. This score is reflective of their ability to metabolize cannabinoids. Scores for the CYP2C9 gene and the CYP3A4 gene can be combined to give a THC compatibility score while scores for the CYP2C19 gene and the CYP3A4 gene can be combined to give a CBD compatibility score. The possible range of scores is 0 to 6 with higher numbers representing a faster predicted cannabinoid metabolism. A scoring matrix for each gene is presented in Table 11.

TABLE 11
Scoring matrix for generating a cannabis compatibility score based on the genetic variants in Table 10.

| CYP2C19 | | | Allele 1 Genotype | | | | | |
|---|---|---|---|---|---|---|---|---|
| | Allele RS ID | | n/a | rs4244285 | rs4986893 | rs41291556 | rs12248560 | rs12769205 |
| | | Star | *1 | *2 | *3 | *8 | *17 | *35 |
| Allele 2 | n/a | *1 | 2 | 1 | 1 | 1 | 3 | 1 |
| Genotype | rs4244285 | *2 | 1 | 0 | 0 | 0 | 2 | 0 |
| | rs4986893 | *3 | 1 | 0 | 0 | 0 | 2 | 0 |
| | rs41291556 | *8 | 1 | 0 | 0 | 0 | 2 | 0 |
| | rs12248560 | *17 | 3 | 2 | 2 | 2 | 4 | 2 |
| | rs12769205 | *35 | 1 | 0 | 0 | 0 | 2 | 0 |

| CYP2C9 | | | Allele 1 Genotype | | | | | |
|---|---|---|---|---|---|---|---|---|
| | Allele RS ID | | n/a | rs1799853 | rs1057910 | rs28371686 | rs7900194 | rs9332239 |
| | | Star | *1 | *2 | *3 | *5 | *8 | *12 |
| Allele 2 | n/a | *1 | 2 | 1 | 1 | 1 | 1 | 1 |
| Genotype | rs1799853 | *2 | 1 | 0 | 0 | 0 | 0 | 0 |
| | rs1057910 | *3 | 1 | 0 | 0 | 0 | 0 | 0 |
| | rs28371686 | *5 | 1 | 0 | 0 | 0 | 0 | 0 |
| | rs7900194 | *8 | 1 | 0 | 0 | 0 | 0 | 0 |
| | rs9332239 | *12 | 1 | 0 | 0 | 0 | 0 | 0 |

| CYP3A4 | | | Allele 1 Genotype | | | | | |
|---|---|---|---|---|---|---|---|---|
| | Allele RS ID | | n/a | rs55785340 | rs72552799 | rs12721629 | rs67666821 | rs35599367 |
| | | Star | *1 | *2 | *8 | *12 | *20 | *22 |
| Allele 2 | n/a | *1 | 2 | 1 | 1 | 1 | 1 | 1 |
| Genotype | rs55785340 | *2 | 1 | 0 | 0 | 0 | 0 | 0 |
| | rs72552799 | *8 | 1 | 0 | 0 | 0 | 0 | 0 |
| | rs12721629 | *12 | 1 | 0 | 0 | 0 | 0 | 0 |
| | rs67666821 | *20 | 1 | 0 | 0 | 0 | 0 | 0 |
| | rs35599367 | *22 | 1 | 0 | 0 | 0 | 0 | 0 |

A THC compatibility score is generated based on genotype data by adding the CYP2C9 and CYP3A4 genotype scores identified in Table 11. A CBD compatibility score is generated based on genotype data by adding the CYP2C19 and CYP3A4 genotype scores identified in Table 11.

Based on their compatibility scores, an individual is recommended a low, medium, high or extra-high daily dose of cannabis. A THC compatibility score is used to recommend doses of all varieties containing THC while the CBD compatibility score is used to recommend doses of varieties containing just CBD such as Avidekel^{MR}.

Individuals scoring less than 2 are recommended a low dose, patients scoring between 2 and 3 inclusive are recommended a medium dose, patients scoring between 4 and 5 inclusive are recommended a high dose and patients scoring greater than 5 are recommended an extra high dose. Scores greater than 4 are only possible on the CBD profile due to the ultra-metaboliser CYP2C19*17 allele. For this reason, there is no extra-high dose recommendation for THC. The dose recommendations for specific cannabis varieties that contain THC are presented in Table 12. The dose recommendations for a specific cannabis variety that contains CBD are presented in Table 13.

### Dosing Considerations

The median average dose of medical cannabis was estimated by taking data on daily prescription amounts and determining the median daily weight of cannabis consumed by a population of subjects using medicinal cannabis. This was converted into a THC weight by taking an average of the THC content of the five most popular THC containing varieties offered by MedReleaf and multiplying this value by the median daily weight of consumed cannabis. A similar process was used to determine the median weight of CBD consumed daily. The medium daily dosage in the following recommendations corresponds to a range around the estimated median daily dose with the low, high and extra high doses defined with reference to this median range.

TABLE 12
Dosage recommendations based on THC compatibility scores
generated using CYP2C9 and CYP3A4 genotypes.

| | Low Daily | | Medium Daily | | High Daily | |
|---|---|---|---|---|---|---|
| | Dose (<2) | | Dose (2-3) | | Dose (>3) | |
| Recommended | 0-343 | mg | 343-573 | mg | 573 | mg+ |
| smoked/vapourised | | | | | | |
| THC dose | | | | | | |
| Cognitiva bud | 0.5-1.5 | g | 1.5-3.0 | g | 3.0 | g+ |
| equivalent | | | | | | |
| Eran Almog bud | 0.5-1.0 | g | 1.0-2.0 | g | 2.0 | g+ |
| equivalent | | | | | | |
| Luminarium bud | 0.5-1.5 | g | 1.5-2.0 | g | 2.0 | g+ |
| equivalent | | | | | | |
| Midnight bud equivalent | 0.5-1.0 | g* | 1.0-2.0 | g* | 2.0 | g+* |
| Nollia bud | 1.0-2.5 | g* | 2.5-3.5 | g* | 3.5 | g+* |
| Equivalent | | | | | | |
| Sedamen bud | 0.5-1.5 | g | 1.5-2.0 | g | 2.0 | g+ |
| equivalent | | | | | | |

*Recommendations for Midnight and Nollia are based on a lower estimated median daily dose of 142 mgTHC. This takes account of the fact that Midnight and Nollia contain a significant amount of CBD which likely contributes to the clinical effects of these two varieties.

TABLE 13
Dosage recommendations based on a CBD compatibility score
generated using CYP2C19 and CYP3A4 genotypes.

| | Low | | Medium | | High | | Extra High | |
|---|---|---|---|---|---|---|---|---|
| | Daily Dose | | Daily Dose | | Daily Dose | | Daily | |
| | (<2) | | (2-3) | | (4-5) | | Dose (>5) | |
| Recommended | 0-195 | mg | 195-325 | mg | 325-400 | mg | 400 | mg+ |
| smoked/vapourised | | | | | | | | |
| CBD dose | | | | | | | | |

| | | | | | |
|---|---|---|---|---|---|
| Avidekel bud | 0.5-1.5 g | 1.5-2.0 g | 2.0 g-3.0 g | 3.0 | g+ |
| equivalent | | | | | |

A variety or varieties of cannabis to be recommended to a patient is determined based on the identification of a medical condition and/or a symptom which they hope to alleviate with medical cannabis. These recommendations are derived from customer surveys of MedReleaf patients including but not limited to those set out in Example 1. Patients were asked which varieties they found most effective at relieving various symptoms and the varieties most associated with relief from specific symptoms form the basis of the following recommendations. A patient may be recommended up to three different varieties of cannabis based on the identified medical condition as shown in Table 14.

TABLE 14
Varietal recommendations for the treatment of
specific medical conditions.

| Symptom | Brand/Variety |
|---|---|
| Anxiety | Midnight |
| | Avidekel |
| | Luminarium |
| Appetite | Cognitiva |
| | Sedamen |
| | Eran Almog |
| Depression | Midnight |
| | Luminarium |
| | Avidekel |
| Inflammation | Avidekel |
| | Midnight |
| | Nollia |
| Pain | Midnight |
| | Avidekel |
| | Sedamen |
| Nausea/Vomiting | Midnight |
| | Sedamen |
| | Avidekel |
| Seizures | Avidekel |
| | Midnight |
| Sleep | Midnight |
| | Sedamen |
| | Eran Almog |

Both a recommended dosage and strain for the treatment of a specific medical condition may therefore be determined. This information can be provided to a patient and/or health care provider in order to facilitate the therapeutically effective use of medicinal cannabis for the treatment of a medical condition.

### Example 3: Treatment of Pain Using Cannabis Varieties High in CBD

Survey data was used to identify a cohort of patients consuming Avidekel™ cannabis (a high CBD variety available from MedReleaf) who reported suffering from pain. Patients were asked whether they had been any changes in their condition since they began to consume MedReleaf cannabis. Patients who responded as having a “Significant/Moderate/Slight Deterioration” or “No Change” were categorized as poor responders while subjects who responded as having a “Significant/Moderate/Slight Improvement” were categorized as good responders.

As shown in Table 15, 7 SNP variants in or near CYP2C9 and CYP2C19 were identified as being associated with a good response (i.e. an improvement) with respect to pain in patients who consumed Avidekel high CBD cannabis. Patients with one or more of the SNP variants identified in Table 15 are therefore expected to respond to the treatment of pain with high CBD cannabis and optionally treatment with Avidekel.

Optionally, a recommended dosage of CBD and/or of Avidekel can be determined for a patient with pain as described herein.

TABLE 15
Identification of SNPs associated with a positive response to treatment of pain with Avidekel (CBD) cannabis.

| snp_id | good-aa | good-ab | good-bb | poor-aa | poor-ab | poor-bb | pval | region | gene | chr | pos | ref | alt |
|---|---|---|---|---|---|---|---|---|---|---|---|---|---|
| rs734969 | 1 | 7 | 1 | 3 | 0 | 2 | 0.019944 | intergenic | CYP2C19, | 10 | 96619086 | A | C |
| | | | | | | | | | CYP2C9 | | | | |
| exm2271332 | 2 | 4 | 3 | 0 | 5 | 0 | 0.115268 | intronic | CYP2C19 | 10 | 96563757 | T | C |
| kgp10780705 | 7 | 2 | 0 | 4 | 0 | 1 | 0.233035 | intronic | CYP2C9 | 10 | 96724061 | T | C |
| kgp12541436 | 1 | 3 | 5 | 0 | 0 | 5 | 0.211072 | intronic | CYP2C9 | 10 | 96714030 | A | G |
| kgp8466518 | 4 | 3 | 2 | 1 | 4 | 0 | 0.217742 | intronic | CYP2C9 | 10 | 96712400 | A | G |
| kgp9307988 | 4 | 4 | 1 | 3 | 0 | 2 | 0.162868 | intronic | CYP2C19 | 10 | 96523130 | T | C |
| rs10518253 | 3 | 4 | 2 | 2 | 0 | 3 | 0.169766 | intronic | CYP2C9 | 10 | 96734339 | A | C |

### Example 4: Treatment of Pain Using Cannabis Varieties Containing THC

Survey data was used to identify a cohort of patients consuming Sedamen™ or Luminarium™ cannabis (varieties of cannabis containing THC available from MedReleaf) who reported suffering from pain. Patients also were asked whether they had been any changes in their condition since they began to consume MedReleaf cannabis. Patients who responded as having a “Significant/Moderate/Slight Deterioration” or “No Change” were categorized as poor responders while subjects who responded as having a “Significant/Moderate/Slight Improvement” were categorized as good responders.

As shown in Table 16, 9 SNP variants in or near CYP2C9, CYP2C19 or CYP3A4 were identified as being associated with a good response (i.e. an improvement) with respect to pain in patients who consumed cannabis containing THC, specifically Sedamen or Luminarium. Using the contingency table set-up shown in Table 16, the chi-squared test was used to calculate the p-value.

Patients with one or more of the SNP variants identified in Table 16 are therefore expected to respond to the treatment of pain with THC, cannabis containing THC and optionally treatment with Sedamen or Luminarium.

Optionally, a recommended dosage of THC, cannabis containing THC and/or of Sedamen or Luminarium can be determined for a patient with pain as described herein.

TABLE 16
Identification of SNPs associated with a positive response to treatment of pain with Sedamen or Luminarium (THC) cannabis

| snp_id | good-aa | good-ab | good-bb | poor-aa | poor-ab | poor-bb | pval | region | gene | chr | pos | ref | alt |
|---|---|---|---|---|---|---|---|---|---|---|---|---|---|
| rs1856908 | 1 | 6 | 2 | 0 | 0 | 3 | 0.06081 | intronic | CYP2C9 | 10 | 96702294 | T | C |
| kgp10378560 | 1 | 7 | 1 | 2 | 1 | 0 | 0.15124 | intronic | CYP2C19 | 10 | 96536066 | A | G |
| kgp10780705 | 8 | 1 | 0 | 2 | 0 | 1 | 0.176694 | intronic | CYP2C9 | 10 | 96724061 | T | C |
| kgp8413369 | 4 | 3 | 2 | 0 | 3 | 0 | 0.135335 | intronic | CYP3A4 | 7 | 99365719 | T | C |
| rs10786189 | 1 | 6 | 2 | 2 | 1 | 0 | 0.144207 | intronic | CYP2C19 | 10 | 96581094 | A | G |
| rs1856908 | 1 | 6 | 2 | 0 | 0 | 3 | 0.06081 | intronic | CYP2C9 | 10 | 96702294 | T | C |
| rs28670611 | 4 | 4 | 1 | 0 | 1 | 2 | 0.123825 | intergenic | CYP2C9, | 10 | 96766934 | A | C |
| | | | | | | | | | CYP2C8 | | | | |
| rs7255901 | 3 | 4 | 2 | 3 | 0 | 0 | 0.135335 | exonic | CYP2C9 | 10 | 96745830 | A | C |
| rs7919934 | 3 | 4 | 2 | 3 | 0 | 0 | 0.135335 | intronic | CYP2C19 | 10 | 96534584 | A | G |

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

Ben Amar M. Cannabinoids in medicine: A review of their therapeutic potential. J Ethnopharmacol 2006; 105(1-2):1-25.
Burckhardt C S, Anderson K L. The Quality of Life Scale (QOLS): reliability, validity, and utilization. Health Qual Life Outcomes 2003; 1(60):1-7.
ElSohly M A, Slade D. Chemical constituents of marijuana: The complex mixture of natural cannabinoids. Life Sci 2005; 78(5):539-48.
Fletcher A, Gore S, Jones D, Fitzpatrick R, Spiegelhalter D, Cox D. Quality of life measures in health care. II: Design, analysis, and interpretation. BMJ 1992; 305(7):1145-8.
Grotenhermen F. The cannabinoid system—a brief review. J Ind Hemp 2004; 9(2):87-92.
Hazekamp A, Tejkalová K, Papadimitriou S. Cannabis: From Cultivar to Chemovar II—A Metabolomics Approach to Cannabis Classification. Cannabis Cannabinoid Res 2016; 1(1):202-15.
Hazekamp A, Fischedick J T. Cannabis—from cultivar to chemovar. Drug Test Anal 2012; 4:660-7.
Huestis M A. Human Cannabinoid Pharmacokinetics. Chem Biodivers 2007; 4(8):1770-804.
Nicholas M K. The pain self-efficacy questionnaire: Taking pain into account. Eur J Pain Blackwell Publishing Ltd; 2007 February; 11(2):153-63.
Russo E B. Taming T H C: potential entourage effects. Br J Pharmacol 2011; 163:1344-64.
Whiting P F, Wolff R F, Deshpande S, Di Nisio M, Duffy S, Hernandez A V, et al. Cannabinoids for Medical Use. Jama 2015; 313(24):2456-73.
Zhornitsky S, Potvin S. Cannabidiol in Humans—The Quest for Therapeutic Targets. Pharmaceuticals 2012; 5:529-52.

1.-44. (canceled)

45. A method of determining a sensitivity of a subject to one or more cannabinoids, the method comprising:
testing nucleic acid from the subject to determine the presence or absence of a genetic variant that modifies the expression and/or activity of one or more of the wild-type CYP2C9, CYP3A4, and CYP2C19 genes, wherein the genetic variant comprises any one or more of the genetic variants identified in Tables 1, 2, and 3.

46. The method according to claim 45, wherein the presence or absence of the genetic variant indicates a sensitivity to cannabidiol (CBD).

47. The method according to claim 45, wherein the presence or absence of the genetic variant indicates a sensitivity to tetrahydrocannabinol (THC).

48. The method according to claim 46, wherein the genetic variant comprises one or more of the genetic variants identified in Tables 1 and 3.

49. The method according to claim 48, wherein the genetic variant modifies the expression and/or activity of the CYP2C19 gene and indicates a decreased, a deleterious, or an increased effect on CBD metabolism.

50. The method according to claim 49, wherein the genetic variant comprises one or more of rs12769205, rs17884712, rs6413438, rs192154563, rs140278421, rs118203757, and rs118203759.

51. The method according to claim 48, wherein the genetic variant modifies the expression and/or activity of the CYP3A4 gene and indicates a decreased or a deleterious effect on CBD metabolism.

52. The method according to claim 51, wherein the genetic variant comprises one or more of rs55785340, rs72552799, rs12721629, rs67666821, rs35599367, rs67784355, rs4986909, rs12721627, rs4987161, and rs138105638.

53. The method according to claim 47, wherein the genetic variant comprises one or more of the genetic variants identified in Tables 1 and 2.

54. The method according to claim 53, wherein the genetic variant modifies the expression and/or activity of the CYP2C9 gene and indicates a decreased or a deleterious effect on THC metabolism.

55. The method according to claim 54, wherein the genetic variant comprises one or more of rs7900194, rs9332239, rs56165452, rs72558187, rs72558190, and rs72558188.

56. The method according to claim 54, wherein the genetic variant modifies the expression and/or activity of the CYP3A4 gene and indicates a decreased or a deleterious effect on THC metabolism.

57. The method according to claim 56, wherein the genetic variant comprises one or more of rs55785340, rs72552799, rs12721629, rs67666821, rs35599367, rs67784355, rs4986909, rs12721627, rs4987161, and rs138105638.

58. The method according to claim 45, wherein the absence of the genetic variant indicates a normal sensitivity to the one or more cannabinoids.

59. The method according to claim 45, further comprising generating a cannabis compatibility score based on the presence or absence of the genetic variant.

60. The method according to claim 59, further comprising comparing the cannabis compatibility score to one or more control scores to determine one or more of a cannabinoid dose, a cannabinoid dosage, and a cannabis variety to prescribe to the subject.

61. An allele-specific polynucleotide suitable for determining the sensitivity of the subject to one or more cannabinoids according to the method of claim 45, wherein the polynucleotide is specific for the genetic variant that modifies the expression and/or activity of one or more of the wild-type CYP2C9, CYP3A4, and CYP2C19 genes.

62. A kit comprising at least one allele-specific polynucleotide defined in claim 61 and at least one further component, wherein the at least one further component is a buffer, a deoxynucleotide triphosphate (dNTP), an amplification primer pair, an enzyme, or any combination thereof.

63. The method of claim 1, further comprising treating the subject for a medical condition with the one or more cannabinoids, wherein the medical condition comprises one or more of anxiety, appetite disorder, depression, inflammation, pain, nausea, vomiting, seizures, and sleep.

64. A method of determining a sensitivity of a subject to one or more cannabinoids for the treatment of pain, the method comprising:
testing nucleic acid from the subject to determine the presence or absence of a genetic variant that modifies the expression and/or activity of one or more of the wild-type CYP2C9, CYP3A4, and CYP2C19 genes, wherein the genetic variant comprises any one or more of the genetic variants identified in Tables 15 and 16.

65. The method according to claim 64, wherein the presence or absence of any one or more of the genetic variants identified in Table 15 indicates a responsiveness of the subject to treating pain with CBD.

66. The method according to claim 64, wherein the presence or absence of any one or more of the genetic variants identified in Table 16 indicates a responsiveness of the subject to treating pain with THC.
